(19) United States
(12) Patent Application Publication
(10) Pub. No.: US 2025/0049998 A1
(43) Pub. Date: Feb. 13, 2025

(54) BLOOD TREATMENT APPARATUS WITH HEAT REGULATION

(71) Applicant: Fresenius Medical Care Deutschland GmbH, Bad Homburg (DE)

(72) Inventors: Lars Breuel, Bad Homburg (DE); Frank Helbig, Bad Homburg (DE); Eckhard Jakob, Bad Homburg (DE)

(21) Appl. No.: 18/720,271

(22) PCT Filed: Dec. 14, 2022
(86) PCT No.: PCT/EP2022/085799

§ 371 Date: Jun. 14, 2024

(30) Foreign Application Priority Data
Dec. 17, 2021 (DE) 10 2021 133 630.9

(51) Int. Cl. A61M 1/16; G16H 40/63

(52) U.S. Cl. CPC A61M 1/1601; A61M 1/1629; G16H 40/63; A61M 2205/3368

(57) ABSTRACT

The present disclosure relates to a blood treatment apparatus with an optional blood pump, a fluid line or a connection site for a fluid line, a pump for conveying treatment liquid through the fluid line, a heating device for heating the treatment liquid within the fluid line and/or within the heating device, a temperature measuring device for detecting, e.g., measuring or determining, the temperature of the treatment liquid if the treatment liquid is present in the fluid line or in the heating device, a storage device for storing temperature values, and a control device for controlling or regulating the aforementioned pumps.

### CROSS-REFERENCE TO RELATED APPLICATIONS

The present application is the national stage entry of International Application No. PCT/EP2022/085799, filed on Dec. 14, 2022, which claims priority to Application No. DE 102021133630.9, filed in the Federal Republic of Germany on Dec. 17, 2021, the contents of which are hereby incorporated by reference thereto.

### TECHNICAL FIELD

The present disclosure relates to a blood treatment apparatus with heat regulation.

### BACKGROUND

Some blood treatment apparatuses apply a treatment liquid to the patient using a pump, often, as in the example of the dialysis apparatus, via the extracorporeal blood circuit. In the case of blood treatment apparatuses, such as dialysis apparatuses, treatment liquid is additionally or alternatively supplied, e.g., to a blood filter perfused by blood or through which blood flows. Since the temperature of the conveyed treatment liquid may in both of the above cases influence the temperature of the blood to be reinfused into the patient, a heating device may be provided for heating or tempering the treatment liquid.

### SUMMARY

The present disclosure relates to a blood treatment apparatus having a heating device and a control device.

In some implementations, the blood treatment apparatus comprises or is connected to an optional blood pump, at least one fluid line, a pump configured to convey treatment liquid through the fluid line, and at least one heating device for heating the treatment liquid within the fluid line and/or within the heating device. In some implementations, the blood treatment apparatus comprises a connection point for connecting the blood treatment apparatus to a fluid line as set forth herein.

In some implementations, the blood treatment apparatus comprises a temperature measuring device, which is provided and/or configured for detecting the temperature of the treatment liquid when the treatment liquid is present in the fluid line or in the heating device. Said detecting may be or encompass for example measuring or determining the temperature of the treatment liquid using at least one temperature sensor.

In some implementations, a storage device for storing amounts of temperature values is also comprised by the blood treatment apparatus. The amounts of temperature values may be or may encompass the values detected by the temperature measuring device.

In some implementations, the blood treatment apparatus further comprises a control device for controlling or regulating the aforementioned pumps, in particular the pump configured to convey treatment liquid. Optionally, the control device is further configured for controlling or regulating the at least one heating device.

In some implementations, the control device is further programmed or configured to prompt the temperature measuring device to measure, or to prompt the measuring of, a single temperature value at each of a plurality of measurement times associated with different measurement intervals, and to determine or detect a prominent, e.g., maximum, minimum, average, etc., temperature value for each of the measurement intervals from the temperature values measured during the measurement interval and to store the prominent temperature values thus determined, or the amounts thereof, in the storage device. In some implementations, the control device is further programmed to read out the stored temperature values, or amounts thereof, from the storage device, and further to read out a preset or predetermined amount of the temperature (for example, the level thereof, such as might be indicated on a display of the temperature measuring device), for example, also from the storage device. The predetermined amount of the temperature indicates which amount the temperature of the treatment liquid, at a predetermined site within the fluid line downstream of the heating device, which is arranged in or at this fluid line, may amount to as a maximum value according to the presetting or may at least amount to as a setpoint.

In some implementations, the control device is further optionally programmed to determine or assume, at least approximately, a cooling of the treatment liquid when the treatment liquid passes towards the predetermined site, which occurs, e.g., between the temperature sensor and the predetermined site.

In some implementations, the control device of the blood treatment apparatus is further programmed to determine, e.g., to calculate, how high the conveyance rate of the pump, which is arranged in or at the fluid line, may be or is allowed to be so that the treatment liquid already present in the fluid line downstream of the heating device and the temperature values of which have been read out, e.g., from the storage device, reaches the predetermined site with a temperature less than or equal to the maximum value for this temperature or amount. Alternatively or additionally, the control device may be programmed to determine the conveyance rate of this pump such that the temperature of the fluid to be conveyed at the predetermined site deviates only insignificantly from a setpoint, is in particular higher than or equal to the setpoint, or lies within a setpoint range.

Further proposed by the present disclosure is a control device designed or programmed as described or disclosed herein.

A digital non-volatile storage medium, particularly in the form of a machine readable carrier, particularly in the form of a diskette, memory card, CD, DVD, EPROM, FRAM (Ferroelectric RAM) or SSD (Solid-State-Drive), particularly with electronically or optically readable control signals, can interact with a programmable computer system of, such that a blood treatment apparatus, e.g., a conventional blood treatment apparatus, a blood treatment apparatus is programmed or reprogrammed to be the blood treatment apparatus described herein and/or such that a control device is programmed or reprogrammed to be the control device described herein.

A computer program product, according to the present disclosure, comprises a volatile or transient program code or one stored on a machine readable carrier or comprises a signal wave which may interact with a programmable computer system such that a blood treatment apparatus, e.g., a conventional blood treatment apparatus, a blood treatment apparatus is programmed or reprogrammed to be the blood treatment apparatus described herein and/or such that a control device is programmed or reprogrammed to be the control device described herein.

A computer program product may be understood as, e.g., a computer program stored on a carrier, an embedded system being a comprehensive system with a computer program (e.g., an electronic device with a computer program), a network of computer implemented computer programs (e.g., client/server-system, a cloud computing system etc.), or a computer on which a computer program is loaded, runs, is stored, is executed or developed.

The term “machine readable carrier” as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

A computer program, according to the present disclosure, encompasses a program code by means of which a blood treatment apparatus is programmed or reprogrammed to be the blood treatment apparatus described herein and/or such that a control device is programmed or reprogrammed to be the control device described herein.

Embodiments according to the present disclosure may comprise one or more of the following features in any combination, unless a person skilled in the art recognizes their combination as technically impossible.

In all of the statements made herein, the use of the expression “may/can be” or “may/can have” and so on, is to be understood synonymously with “preferably is” or “preferably has,” and so on respectively, and is intended to illustrate embodiments according to the present disclosure.

Whenever numerical words are mentioned herein, a person skilled in the art shall recognize or understand them as indications of a numerical lower limit. Unless stated otherwise, a person skilled in the art shall comprehend the specification for example of “one” (also “a/an”) as encompassing “at least one”. This understanding is also equally encompassed by the present disclosure as the interpretation that a numeric word, for example, “one” (also “a/an”) may alternatively mean “exactly one”, wherever this is evidently technically possible for a person skilled in the art. Both understandings are encompassed by the present disclosure and apply herein to all used numerical words.

Whenever reference is made herein to spatial indications, such as “top”, “bottom”, “left” or “right”, the skilled person understands this to mean the arrangement in the figures attached hereto and/or in the state of use. “Bottom” is closer to the center of the earth or the lower edge of the figure than “top”.

Advantageous developments of the present disclosure are each also subject-matter of the dependent claims and embodiments.

Whenever an embodiment is mentioned herein, that embodiment is an exemplary embodiment according to the present disclosure.

When disclosed herein that the subject matter according to the present disclosure comprises one or several features in a particular embodiment, it is also respectively disclosed herein that the subject matter according to the present disclosure does, in some embodiments, likewise according to the present disclosure, explicitly not comprise this or these features, for example, in the sense of a disclaimer. Thus, for every embodiment mentioned herein it applies that the converse embodiment, e.g., formulated as negation, is also disclosed.

The control device is, in some embodiments, configured to automatically prompt or to let execute one, several or all of the method steps described herein.

If method steps are mentioned herein, the control device or the blood treatment apparatus may comprise corresponding devices which names can be respectively based on the designation of the corresponding method step (e.g., “to determine” as a method step and “determining device” for the device, etc.) and which devices may also be part of the apparatuses according to the present disclosure or connected thereto in signal communication in order for the devices to be controlled by apparatuses according to the present disclosure accordingly.

Whenever programed or configured is mentioned herein, then these terms may in some embodiments be interchangeable.

When reference is made herein to a signal communication or communication connection between two components, this may be understood to mean a connection that exists in use. Likewise, it may be understood herein that there is a preparation for such a signal communication (wired, wireless, or otherwise implemented), for example, by a coupling of both components, such as by pairing, etc.

Pairing is to be understood as a process that takes place in connection with computer networks in order to establish an initial link between computer units for the purpose of communication. The best-known example of this is the establishment of a Bluetooth connection, by which various devices (e.g., smartphone, headphones) are connected to one another. Pairing is sometimes also referred to as bonding.

The control device may prompt the execution of all or substantially all of the method steps. A method described herein may be carried out substantially or completely by the control device. The method may be partially carried out by the control device, in particular those steps which do not require or relate to human intervention and/or provision may be carried out by the control device. The control device may serve purely as a control device or also as a closed-loop control device.

In some embodiments, the control device is present in or on the blood treatment apparatus, for instance together with further components or devices of the blood treatment apparatus in a common housing of the blood treatment apparatus.

In some embodiments of the blood treatment apparatus, the pump for conveying the treatment liquid is a dialysis liquid pump, which is arranged in or on a dialysis liquid inlet line in order to convey dialysis liquid as treatment liquid into a dialysis liquid chamber of a blood filter or dialyzer, wherein the blood filter comprises, in addition to the dialysis liquid chamber, a blood chamber separated therefrom by a membrane.

In some embodiments, the pump is a substitute fluid pump arranged in or on a substitute fluid line to convey substitute fluid as a treatment liquid to the extracorporeal blood circuit of the blood treatment apparatus (either in pre- or post-dilution) at addition sites provided for this purpose.

In some embodiments, the predetermined site is the dialysis liquid chamber of the blood filter or an inlet to the blood filter or to the dialysis liquid chamber, respectively.

In some embodiments, the predetermined sit is an addition site for treatment liquid as a substitute fluid into the extracorporeal blood circuit.

In some embodiments, the length or duration of the measurement intervals is constant, for example 15 seconds each. The length or duration may be stored in the storage device.

In some embodiments, the amounts of the quantities of treatment liquid respectively conveyed in the fluid line in the measurement intervals are stored, e.g., in the storage device.

In some embodiments, the amounts of the quantities of treatment liquid respectively conveyed in the fluid line in the measurement intervals are stored together with the detected prominent temperature value and a designation for identifying the associated measurement interval for which the temperature value was detected, respectively.

In some embodiments, the cooling of the treatment liquid as the treatment liquid passes between the temperature measuring device or the temperature sensor thereof and the predetermined site is determined or at least approximately determined or assumed by calculation(s) using one or more tables, e.g., look-up tables.

In some embodiments, the storage device is or comprises a history buffer, a ring buffer, a ring memory or the like, in which the detected temperature values, are stored or are to be stored.

The storage device may be equipped with, e.g., 30 elements. Each element optionally includes, for a measurement interval (of a period of, e.g., 15 seconds), the maximum or otherwise prominent temperature value measured by the temperature measurement device or by the temperature sensor thereof during that period, and the volume of treatment liquid conveyed during that interval, provided the latter has not been kept, e.g., constant.

In some embodiments, the control device is programmed to limit or otherwise control or regulate the conveyance rate of the pump for conveying the treatment liquid such that the temperature of the treatment liquid conveyed by the pump, when the treatment liquid has been conveyed to the predetermined site, does not exceed a predetermined temperature value previously judged to be harmless to the patient, e.g., 41° C., at that predetermined site.

In some embodiments, the control device is programmed in order to correct the detected temperature values or their amounts downward by a correction factor, particularly after they have been stored, for periods during which the pump for conveying the treatment liquid has or is stopped and thus is not conveying.

In this way, e.g., a virtual minimum flow is assumed when the pump is stopped in order to simulate cooling of the treatment liquid in the fluid line.

In some embodiments, the blood treatment apparatus is embodied as a dialysis apparatus for hemodialysis apparatus, hemofiltration apparatus, hemodiafiltration apparatus, in particular as an apparatus for the acute renal replacement therapy, the chronic renal replacement therapy or the continuous renal replacement therapy (CRRT).

In some embodiments, the blood treatment apparatus is connected to or equipped with a blood tubing set and/or a fluid line for the treatment liquid which is provided or approved for pediatric blood treatment. In some embodiments, the blood treatment apparatus is connected to or equipped with a blood tubing set and/or a fluid line for the treatment liquid which is provided or approved for adult blood treatment.

In some embodiments, a measurement interval is a time interval, e.g., of predetermined length, which may also be the length of the other time intervals under consideration, within which the temperature is repeatedly determined in a plurality of measurements in order to determine the herein-disclosed prominent temperature value for that time interval.

In some embodiments, it may be provided to determine a superordinate prominent temperature value for the treatment liquid for the complete volume (corresponding to the totality of the partial volumes) between the temperature sensor and the predetermined site, in other words, a prominent temperature value of that volume which has already passed the temperature sensor but has not yet passed the predetermined site. The complete volume could be composed of, e.g., the volume of a heating bag downstream of the temperature measuring device or temperature sensor thereof and the volume of the fluid line connected to the heating bag, e.g., up to the predetermined site. For example, a volume, of 30 ml, within the heating bag but downstream of the temperature measuring device and a volume, of 5 ml, of the fluid line connected to the heating bag would accordingly result in a complete volume of 35 ml. In order to determine the superordinate prominent temperature value of the treatment liquid or amount, e.g., related to the complete or total volume or lumen, a superordinate prominent, e.g., maximum, temperature value for the complete volume may be further detected or determined, for example, from the prominent temperature values determined for each measurement interval. The prominence of this temperature value may, but need not, be the same prominence as when determining the prominent temperature values of the individual measurement intervals. In other words, if, for example a maximum temperature value has been determined from the measured temperatures as a prominent temperature value for each of the individual measurement intervals, then, analogously to this, a maximum temperature value may in turn be determined as a superordinate prominent temperature value from these prominent temperature values for the complete volume between the temperature sensor and the predetermined site. However, this is not to be understood as limiting; for example, an average or the like could alternatively be determined as a superordinate prominent temperature value, which in turn, however, was also determined from a plurality of maximum temperature values, etc., or vice versa.

Some or all embodiments may have one, several or all of the advantages mentioned above and/or below.

If, as may be provided, the heating capacity of the heating device is subject to temperature-based control or limitation, it may be ensured that the temperature at the predetermined site does not exceed a maximum temperature and/or does not fall below a setpoint temperature. By an appropriate adjustment of the conveyance rate of the pump for conveying the treatment liquid with regard to the above temperature values, the patient may, hence, be protected from the consequences of using a treatment liquid having an unsuitable temperature.

In some embodiments, determining this maximum allowable liquid flow, e.g., dialysate flow or substitute fluid flow, and knowledge of the temperature of the liquid in the area between the heating device and the predetermined site is possible without the need for an additional sensor.

If the treatment liquid is heated during dialysis treatment, the temperature of the liquids at predetermined sites, in particular at the addition site towards the blood system (e.g., at the inlet of the dialyzer or at an addition site for substitute fluid) should correspond to the manufacturer's specifications. In this, it may be sufficient to measure the temperature only in the heating device, where a temperature measuring device or a temperature sensor is usually already provided, and not also at the predetermined point, where usually no further temperature measuring device is present.

Nevertheless, the temperature or a heat loss of the treatment liquid at or until the temperature reaches the predetermined site is known, and the flows of the treatment liquid can be regulated accordingly.

The treatment liquid heated in the heating devices may thereby noticeably cool down before the corresponding predetermined site is reached. The heat loss hereby may depend on four factors: ambient temperature; air circulation; temperature of the treatment liquid to be heated in the bag; and set treatment liquid flow.

In order to counteract this heat loss (cooling down), on the way to the predetermined site, in some embodiments the setpoint temperature (and thus the setpoint temperature for the control algorithm of the heating) is advantageously adjusted, e.g., increased. Said adjustment is possible when being aware of the heat losses and with regard to the fact that a—viewed subsequently—however possibly too high a temperature was being selected at the outlet of the heating bag, it is ultimately harmless to the patient when using the regulation of the flow velocity within the fluid line described herein by regulating the conveyance rate of the fluid pump, since in particular the flow velocity in the fluid line has a strong influence on how much the treatment liquid cools down on the way to the predetermined site. For example, at low flow velocities, the temperature of the treatment liquid at the outlet of the heating device is significantly higher than at higher flow velocities, amongst others, because of the correspondingly longer dwell time in the fluid line at lower flow velocities.

Conversely, a sudden increase in the flow velocity would result in a significantly higher temperature at the predetermined site than the desired temperature and would possibly even endanger the patient. By using the present disclosure, the permissible flow may, due to the maximum temperature in the area following the temperature sensor, advantageously be calculated or may be, e.g., approximately determined, for instance by looking in a look-up table, with which permissible flow there is still a permissible temperature value of the treatment liquid at the predetermined site via/after the cooling down of the treatment liquid within the fluid line. As a result, the liquid may further be treated with the maximum possible flow and thus, advantageously, the greatest possible treatment effectiveness may be achieved. Throttling the heating device or reducing the treatment liquid flow, as was previously necessary to achieve a desired temperature value for the treatment liquid at the predetermined site, and which led to reduced treatment effectiveness, may thus be largely avoided or at least advantageously controlled.

### BRIEF DESCRIPTION OF THE DRAWINGS

In the following, the present disclosure is purely exemplarily described with reference to the accompanying figures in which identical reference numerals designate same or similar components. The following applies:

FIG. 1 shows a schematically simplified flow diagram of a continuous veno-venous hemodiafiltration (CVVHDF) with post-dilution of a blood treatment apparatus in an exemplary embodiment;

FIG. 2 shows components of a blood treatment apparatus in an exemplary embodiment;

FIG. 3 shows a schematically simplified fluid line diagram of a blood treatment apparatus in a further exemplary embodiment.

### DETAILED DESCRIPTION

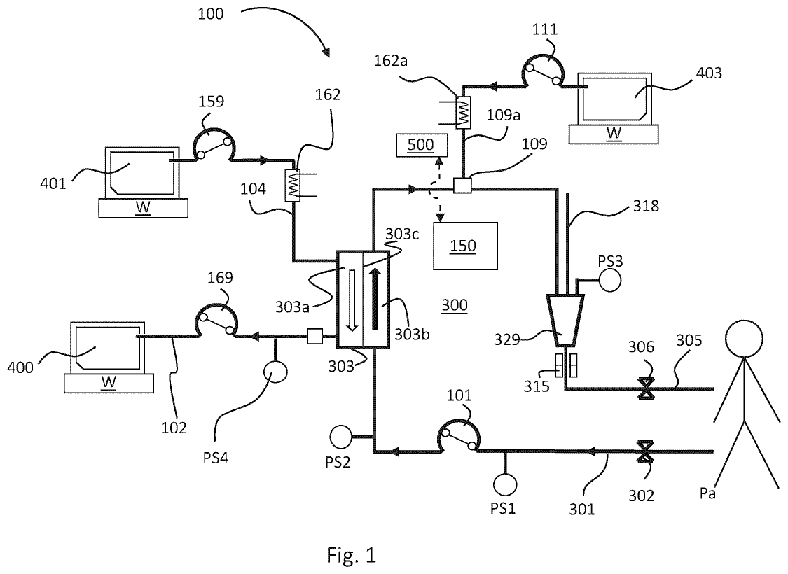

FIG. 1 shows a schematically simplified flow diagram of a continuous veno-venous hemodiafiltration (CVVHDF) with post-dilution of a blood treatment apparatus 100 in a first exemplary embodiment.

The blood treatment apparatus 100 (shown in FIG. 1 only by individual, schematically highly simplified components) comprises a blood pump 101 for conveying blood along, through or within, a blood tubing set or an extracorporeal blood circuit 300, which in turn optionally comprises a venous bubble trap 329.

The optional venous bubble trap 329 may comprise a deaeration device 318 and may be in fluid communication with the pressure sensor PS3. The arrangement of FIG. 1 comprises an optional air bubble detector 315 for detecting air and/or blood. Here, the air bubble detector 315 is arranged downstream of the venous bubble trap 329.

The blood tubing set 300 has an arterial line section 301 (also referred to as first line, arterial patient line, or blood withdrawal line) interacting with an arterial patient tube clamp 302 of the blood treatment apparatus 100 and may be closed by said patient tube clamp 302. The blood tubing set 300 further comprises, or is connected to, connectors for an arterial connection needle or a connection for a central venous catheter.

Further, the blood tubing set 300 may be closed at a venous line section 305 (also referred to as venous patient line, blood return line, or second line) by a venous patient tube clamp 306 of the blood treatment apparatus 100, and has, or is connected to, connectors for a venous connection needle or a connection for a central venous catheter. The optional connection needles are provided to be connected to the blood circuit of a patient Pa.

In the example of FIG. 1, an embodiment of a control device 150 is outlined, which is configured to control or regulate the blood treatment apparatus 100, in particular (to control or regulate) the blood pump 101, a first flow pump 159 and/or a substitute pump 111.

The extracorporeal blood circuit or the blood tubing set 300 comprises, in the example of FIG. 1, further a blood filter 303 or dialyzer the blood chamber 303b of which is connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, e.g., spent dialysis liquid, leading away from the dialysis liquid chamber 303a. For this purpose, suitable connectors are used on the dialysis liquid inlet line 104 or on the dialysate outlet line 102 on the one hand and on the dialysate ports on the other hand, which may be connected to each other, in particular releasably.

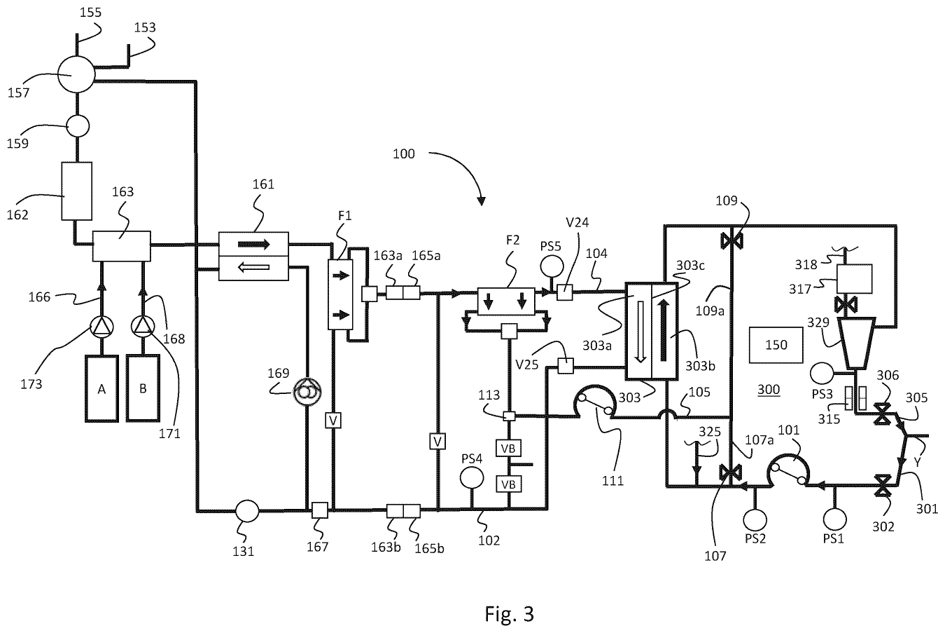

Dialysis liquid chamber 303a and blood chamber 303b are separated from each other by a mostly semi-permeable membrane 303c. Blood and dialysis liquid are mostly guided through the blood filter 303 by the counter current principle. The blood is purified in the blood filter 303. The semi-permeable membrane represents the separation between the blood side with the extracorporeal blood circuit 300 and the machine side with the dialysis liquid circuit or dialysate circuit, which is shown to the left of membrane 303c in FIG. 1 and FIG. 3. In relation to FIG. 3, the machine side is described in a further embodiment in more detail.

In the example of FIG. 1, fresh dialysis liquid (as an example for a treatment liquid) is conveyed by a first flow pump 159 out of a dialysis liquid source 401, here a bag with dialysis liquid, to the dialysis liquid chamber 303a via the dialysis liquid inlet line 104 (as an example for a fluid line). The dialysis liquid may be heated or brought to a predetermined temperature by a heating device 162.

The dialysate, which is also referred to as effluent and which is, or comprises, spent dialysis liquid, possibly enriched with filtrate, leaves the dialysis liquid chamber 303a of the blood filter 303 via the dialysate outlet line 102, optionally conveyed by a second flow pump 169.

In the example of FIG. 1, the effluent is first collected in an optional effluent bag 400 to be subsequently discarded into a sink or into another suitable drain.

The scales W are used to determine the respective amount of dialysis liquid supplied or the respective amount of effluent (also: filtrate) discharged. The scales W, or their measurements values, serve for balancing.

Using the postdilution valve 109, the extracorporeal blood circuit 300 is supplied with substitute fluid (as further example for a treatment liquid) from a substitute fluid source 403, herein a substitute fluid bag. This is conveyed by a substitute fluid pump 111 arranged in or on the line 109a (as further example for a fluid line) associated with the post-dilution valve; optionally the substitute fluid is heated or brought to a predetermined temperature in a heating device 162a. By using the scale W, the supplied amount of substitute fluid may be determined. The values provided by the scale W may also serve for balancing.

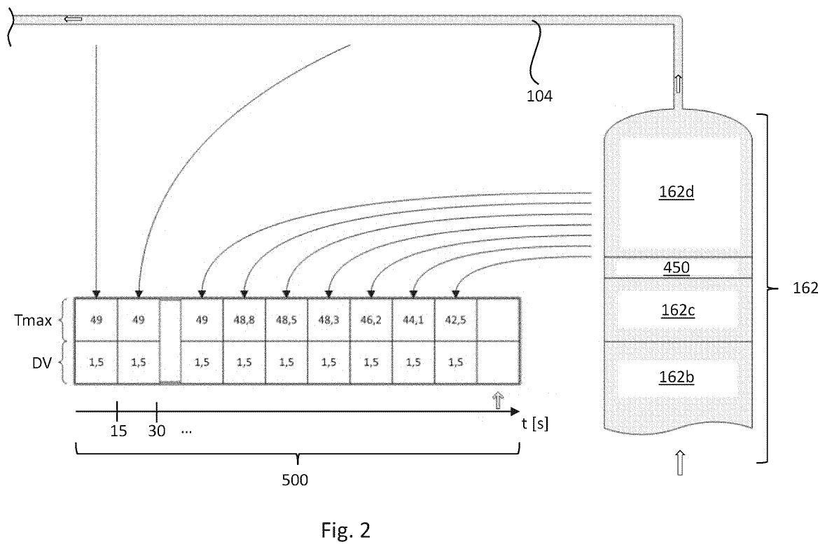

By using the control device 150, a temperature measuring device 450 disposed at any of the heating devices 162, 162a may be prompted to determine, e.g., a maximum temperature value Tmax as a prominent temperature value for a measuring interval, respectively, and to store the value in a storage device 500. This will be described in more detail with regard to FIG. 2.

The control device 150 may be further programmed or configured to read out the stored temperature values from the storage device 500.

In addition, the control device 150 may be configured in order to read out, from the storage device 500, a preset or predetermined amount of the temperature which the dialysis liquid is allowed to maximally have-according to a presetting defined as maximum value-at a predetermined site within the dialysis liquid inlet line 104 downstream of the heating device 162, e.g., at the inlet of the dialysis liquid inlet line 104 in the dialysis liquid chamber 303a of the dialyzer 303.

Alternatively or additionally, the control device 150 may be configured in order to read out, from the storage device 500, a preset or predetermined amount of the temperature which the substitute fluid is allowed to maximally have—according to a presetting defined as maximum value—at a predetermined site within the substitute fluid line 109a downstream of the heating device 162a, e.g., the addition site 109 for substitute fluid in the extracorporeal blood tubing set 300.

This applies analogously to setpoints which the temperature values of the treatment liquid should minimally have at the predetermined point, respectively.

In FIG. 2, there is described in more detail how to estimate a cooling of the dialysis liquid as the dialysis liquid passes between the temperature measuring device 450 or the temperature sensor thereof and the predetermined site as well as how to determine how high the conveyance rate of the pump 159, 111 is maximally allowed to be, respectively, such that the dialysis liquid already present downstream of the heating device 162 in the dialysis liquid inlet line 104 or the substitute fluid already present downstream of the heating device 162a in the substitute fluid line 109a reaches the respective predetermined sites having a maximum permissible temperature.

Further pressure measuring devices PS1, PS2, PS4 may optionally be arranged together or independently of each other on the various lines.

FIG. 2 shows, on the right, the heating device 162 of a blood treatment apparatus 100 in an exemplary embodiment. In the example of FIG. 2, the heating device 162 serves to heat the dialysis liquid being as an example of a treatment liquid within a fluid line, in this case the dialysis liquid inlet line 104. The treatment liquid of FIG. 2 is conveyed by a first flow pump 159 through the heating device 162 and the fluid line.

The heating device 162 is optionally shown as a heating bag, which comprises a heated area 162b, optionally an unheated area 162c, a temperature measuring device 450, here exemplarily in the form of a ring sensor, and a volume 162d, for example of about 30 ml, downstream of the ring sensor.

The temperature measuring device 450 serves to detect, e.g., measure or determine, the temperature of the dialysis liquid when the dialysis liquid is present in the heating device 162 or when the dialysis liquid flows past the temperature measuring device 450 or the temperature sensor thereof, respectively.

By using the control device 150, the temperature measuring device 450 is prompted to determine a maximum temperature value Tmax at a plurality of measuring times, shown here along the time axis t, respectively, within a specific measuring interval (or time interval), here for example 15 seconds, and to store Tmax in the storage device 500. This is shown with exemplary temperature values Tmax to the left of the heating device 162. The storage of the temperature values Tmax, or their amounts, may also be referred to herein as a history buffer.

The control device 150 may be further programmed or configured to read out the stored temperature values from the storage device 500.

Further, a preset or predetermined temperature value which the treatment liquid, here dialysis liquid, is allowed to maximally have or should not exceed, at a predetermined site within the fluid line, here the dialysis liquid inlet line 104, downstream of the heating device 162, as a maximum value according to the presetting, may be stored in the control device 150 or the storage device 500, in order to be read out from the storage device 500, in order to be read out from the memory storage 500 via the control device 150. The same applies analogously to setpoints of the temperature, in particular the minimum temperature, or a setpoint range for the temperature at the predetermined site.

The storage device 500 may be provided, e.g., for storing a temperature history. The temperature history may in turn be, or encompass, a predetermined number of detected prominent temperature values, hereinafter referred to purely by way of example as maximum temperature values Tmax respectively detected during the measurement interval. Instead of maximum temperature values, the middle, average, lowest or other predetermined prominent temperature values could also be detected and stored.

In some embodiments, the storage device 500 is programmed to store, for example, 30 prominent temperature values Tmax, wherein each stored temperature value Tmax is, e.g., the maximum temperature measured in a fixed predetermined measurement interval, e.g., 15 seconds, and wherein these measurement intervals may each one following the other, e.g., sequential.

Based on the predetermined measurement interval, the temperature value Tmax is indirectly assigned to a specific partial volume DV of the dialysis liquid along the path of the liquid through the fluid line, wherein the model conception assumes an ideal plug flow, e.g., that the flow velocity is the same almost everywhere in the flow cross-section of the fluid line, here the dialysis liquid inlet line 104. Thus, in the example of FIG. 2, the temperature history, covering a period of 30×15 s=450 s, is stored.

For example, once the 30 temperature values Tmax, or other prominent temperature values, are stored in the storage device 500, the oldest temperature value Tmax is overwritten by a new temperature value, e.g., in the next measurement interval. In some embodiments, the storage elements of the storage device 500 are thus continuously overwritten such that only the most recent 30 recorded, prominent temperature values are stored in chronological order of their occurrence.

In addition, in the measuring interval—measurable, e.g., due to the pump rotation—the amount of the dialysis liquid volume DV conveyed, also referred to herein as the amount of the volume of treatment liquid, may be stored in the storage device 500.

Based on the temperature values Tmax of the temperature history stored in the storage device 500, it can be determined “in advance” in the model conception when a certain partial volume with a certain temperature will reach the predetermined site. Until the predetermined site is reached, cooling takes place along the conveying path, and this may hereby be taken into account.

The arrows shown represent an association of the treatment liquid present in the dialysis liquid inlet line 104 with the detected, prominent temperature values Tmax and the volume DV of treatment liquid, here dialysis liquid, delivered in the measuring interval.

In the example shown here for measuring temperature values Tmax using the temperature measuring device 450, the actual temperature values therein decrease over time, while the volume DV of treatment liquid delivered in the measurement interval remains the same. However, this may not be the case in some embodiments. Thus, embodiments in which the volume DV of treatment liquid delivered in the measurement interval varies, e.g., by a conveyance rate determined according to the present disclosure, are also encompassed by the present disclosure. Additionally or alternatively, the present disclosure also encompasses embodiments in which the temperature measured by the temperature measuring device 450 remains stable.

If the first flow pump 159 stops within an exemplary measurement interval of, e.g., 15 seconds, the measurement interval, e.g., continues when the first flow pump 159 restarts.

The internal volume of a pediatric dialysis liquid inlet line 104 between the outlet of the heating bag and the inlet of the dialysis liquid chamber 303a of the dialyzer 303 is, for example, approximately 5 ml. In the case of a dialysis liquid inlet line 104 for treating an adult, this volume is approximately 10 ml. These quantities may be used in the calculation of a heat loss.

The heat losses due to which the dialysis liquid cools down while the dialysis liquid is being pumped from the heating bag 62 to the inlet of the dialysis liquid chamber 303a of the dialyzer 303 may be taken into account in different ways in such a temperature-based control or limitation of the conveyance rate of the first flow pump 159 (also dialysis liquid pump, see FIG. 1 or FIG. 3). For example, a calculation model for the heat balance can be created. The calculation model calculates in particular the heat losses on the dialysis liquid inlet line 104. This is done, for example, depending on the conveyance rate of the first flow pump 159, the properties of the dialysis liquid inlet line 104, other boundary or marginal conditions such as the ambient temperature, and/or the temperature values Tmax stored in the storage device 500 as or for the temperature history. The conveyance rate of the first flow pump 159 may be determined based on this calculation such that the temperature at the predetermined site, for example at the inlet of the dialysis liquid chamber 303a of the dialyzer 303, does not exceed the maximum permissible temperature on the one hand, and does not fall significantly below the setpoint temperature on the other hand.

In some embodiments of the present disclosure, in order to limit the programming effort of such calculation models for the heat balance while taking into account the cooling of the dialysis liquid flowing in the dialysis liquid inlet line 104, associations of the values of (i) the maximum temperature at the predetermined site, (ii) the maximum measured temperature value at the temperature measuring device 450, and (iii) the conveyance rate used by the dialysis liquid pump may have been determined empirically in test series and stored in a look-up table in the software.

In some embodiments of the present disclosure, in order to determine the prominent, e.g., maximum, temperature value within a measurement interval when passing the temperature measuring device 450, here the ring sensor, over, e.g., 15 seconds, the temperature of the treatment liquid is measured several times in order to determine the prominent, e.g., maximum, temperature value Tmax from these measured temperature values. This is carried out for a series of successive measurement intervals, of which the correspondingly conveyed partial volumes of the treatment liquid, that were conveyed, e.g., in these 15 seconds, are known.

For setting the conveyance rate of the first flow pump 159, here the dialysis liquid pump, the control device 150 determines the maximum allowable conveyance rate of said flow pump 159 in light of the detected temperature values and controls the pump accordingly. The determination may be carried out, e.g., based on a look-up table for the maximum permissible temperature at the predetermined site, which can be stored in the storage device, in connection with the prominent, here maximum, temperature value which was measured at the temperature measuring device 450 in a specific measuring interval. The inventors were able to show that the maximum temperature of the dialysis liquid at the predetermined site can be reliably maintained with sufficient accuracy using the control system.

In some embodiments, when the first flow pump 159 does not convey, a “virtual” dialysis liquid flow rate of 5 ml/min is assumed in the software and also the temperature at the temperature measuring device 450 is stored in the data memory for the temperature history in order to be able to account for the cooling.

Thus, advantageously additionally, a temperature-based control or limitation of the conveyance rate of the first flow pump 159 is added to the control of the heating capacity of the heating device 162 based on the temperature values Tmax measured by the temperature measuring device 450 is in order not to exceed a maximum temperature at the predetermined site and not to fall below a setpoint temperature.

All what is stated herein with respect to the heating device 162, which is arranged in the dialysis liquid inlet line 104, applies analogously to heating devices 162a, which may be arranged in a substitute fluid line 105, 107a, 109a (see the description of FIG. 1 and FIG. 3). In these embodiments, a predetermined site is, for example, an addition site 107, 109 for dialysis liquid as a substitute fluid in pre- or post-dilution into the extracorporeal blood circuit 300, the conveying pump is accordingly a substitute fluid pump 111 (see FIG. 1 and FIG. 3).

FIG. 3 shows, schematically simplified, a fluid line structure of a blood treatment apparatus 100 in another exemplary embodiment.

Reference is made herein to the description of FIG. 1. In the following, particularly the differences from FIG. 1 will be discussed.

The blood treatment apparatus 100 is connected to an extracorporeal blood circuit 300, which may be connected to the vascular system of the patient Pa for executing a treatment using double-needle access, or using, e.g., an additional Y-connector (reference numeral Y), as is shown in FIG. 3 using single-needle access. The extracorporeal blood circuit 300 may be present, optionally in sections thereof, in or on a blood cassette.

Pumps, actuators and/or valves in the area of the extracorporeal blood circuit 300 are connected to the blood treatment apparatus 100 or to a control device 150, e.g., comprised by the blood treatment apparatus 100, which can be a closed-loop control device. The statements made herein regarding the control device 150, in particular with regard to FIG. 1 and FIG. 2, also apply analogously to the control device 150 of FIG. 3.

Analogously to FIG. 1, the optional blood pump 101 is provided in or on the arterial line 301. The substitute fluid pump 111 is in this embodiment arranged in or on a substitute fluid line 105 and may be fluidically connected to the dialysis liquid inlet line 104. Using the substitute fluid pump 111, substitute fluid may be introduced by pre-dilution, via a pre-dilution valve 107, or by post-dilution, via a post-dilution valve 109, via associated lines 107a or 109a into line sections, for example into the arterial line section 301 or into the venous line section 305 (here between a blood chamber 303b of a blood filter 303 and a bubble trap 329, here a venous air separation chamber) of the blood tubing set 300.

The arrangement of FIG. 3 further encompasses an optional valve V24 arranged in the dialysis liquid inlet line 104 upstream of the blood filter 303, but downstream of a likewise optional pressure sensor PS5. The arrangement of FIG. 3 further encompasses an optional valve V25 arranged in the dialysate outlet line 102 downstream of the blood filter 303, however upstream of the, optional pressure sensor PS4.

The arrangement in FIG. 3 encompasses an air bubble detector 315 for detecting air and/or blood; one or two pressure sensors PS1 (upstream of the blood pump 101) and PS2 (downstream of the blood pump 101, which measures the pressure upstream of the blood filter 303 (“pre-hemofilter”)), e.g., at the points shown in FIG. 3. Further pressure sensors may be provided, e.g., pressure sensor PS3 downstream of the venous bubble trap 329.

An optional single-needle chamber 317 is used in FIG. 3 as a buffer and/or compensating reservoir in a single-needle procedure in which the patient is connected to the extracorporeal blood circuit 300 using only one connecting needle having the two blood lines 301, 305.

A Heparin addition site 325 may optionally be provided.

On the machine side, left in FIG. 3, a mixing device 163 is shown, which provides a predetermined mixture for the respective solution from the containers A (for A concentrate via concentrate supply 166) and B (for B concentrate via concentrate supply 168) for use by the blood treatment apparatus 100. The solution contains water from the water source 155 (on-line, e.g., as reverse osmosis water or from bags), wherein the water is heated e.g. in the heating device 162.

A pump 171, which can be referred to as concentrate pump or sodium pump, is fluidically connected to the mixing device 163 and a source of sodium, for example the container A, and/or conveys out of it. An optional pump 173, which is assigned to container B, for example for bicarbonate, can be seen.

Furthermore, FIG. 3 shows a drainage 153 for the effluent. An optional heat exchanger 157 and the first flow pump 159, which is suitable for degassing, complete the arrangement shown.

The pressure sensor PS4 disposed downstream of the blood filter 303 on the water side, in some cases upstream of an ultrafiltration pump 131 in the dialysate outlet line 102, may be provided for measuring the filtrate pressure or membrane pressure of the blood filter 303.

Blood leaving the blood filter 303 flows through an optional venous blood chamber 329, which may comprise a deaeration device 318 and may be in fluid communication with the pressure sensor PS3.

By using the device for on-line mixing of the dialysis liquid, a variation of sodium content, controlled by the control device 150, is possible within certain limits. For this purpose, in particular the measured values determined by the conductivity sensors 163a, 163b may be taken into account. Should an adjustment of the sodium content of the dialysis liquid (sodium concentration) or of the substitute fluid turn out to be necessary or desired, this can be done by adjusting the conveyance rate of the sodium pump 171.

In addition, the blood treatment apparatus 100 comprises means for conveying fresh dialysis liquid and dialysate. A first valve may be provided between the first flow pump 159 and the blood filter 303, which first valve opens or closes the inflow towards the blood filter 303 at the inlet side. The second, optional flow pump 169 is provided, e.g., downstream of the blood filter 303 and conveys dialysate to the drainage 153. A second valve may be provided between the blood filter 303 and the second flow pump 169 at the outlet side, which second valve opens or closes the outflow.

Furthermore, the blood treatment apparatus 100 optionally comprises a device 161 for balancing the flow flowing into and out of the dialyzer 303 on the machine side. The device 161 for balancing can be arranged in a line section between the first flow pump 159 and the second flow pump 169.

The treatment apparatus 100 further comprises means, such as the ultrafiltration pump 131, for the precise removal of a volume of liquid, as predetermined by the user and/or by the control device 150, from the balanced circuit.

Sensors such as the optional conductivity sensors 163a, 163b serve to determine the conductivity, which in some embodiments is temperature-compensated, as well as the fluid flow upstream and downstream of the dialyzer 303.

Temperature sensors 165a, 165b may be provided as one or a plurality thereof. Temperature values supplied by them may be used by the present disclosure to determine a temperature-compensated conductivity.

A connection for an optional compressed air device, for example a compressor, may be additionally provided on the machine side upstream of the blood filter 303 and/or elsewhere.

A leakage sensor 167 is optionally provided, and may alternatively be provided at another site.

Further flow pumps in addition to or alternative to, e.g., flow pump 169 may also be provided.

A number of optional valves are each denoted with V in FIG. 3. By-pass valves are denoted with VB.

In some embodiments, the control device 150 determines the electrolyte and/or liquid balance based on the measured values of the aforementioned optional sensors.

Even when using non-pure water, the filter F1 exemplarily serves herein to generate sufficiently pure dialysis liquid by the mixing device 163, which then flows through the blood filter 303, e.g., using the countercurrent principle.

A filter F2 exemplarily serves herein to generate sterile or sufficiently filtered substitute fluid from the sufficiently pure dialysis liquid leaving the first filter F1, by filtering, e.g., pyrogenic substances. This substitute fluid may then be safely added to the extracorporeally flowing blood of the patient and thus ultimately to the patient's body.

Filters F1 and F2 can be provided connected in series.

The blood treatment apparatus 100 is shown in FIG. 3 optionally as a device for hemodiafiltration. However, hemodialysis apparatuses are also covered by the present disclosure, although not specifically represented in a figure.

The present disclosure is not limited to the embodiments described herein; they only serve for illustration.

The arrows shown in the figures generally indicate each the flow direction.

Heating devices with associated temperature measuring devices as shown in FIG. 1 or 2 and disclosed herein may be provided in the blood treatment apparatus 100 of FIG. 3.

### LIST OF REFERENCE NUMERALS

100 blood treatment apparatus
101 blood pump
102 dialysate outlet line
104 dialysis liquid inlet line
105 substitute fluid line
107 pre-dilution valve
107a line associated with the pre-dilution valve
109 post-dilution valve
109a line associated with the post-dilution valve
111 substitute fluid pump
131 ultrafiltration pump
150 control device
153 drainage or discharge
155 water source
157 heat exchanger
159 first flow pump
161 device for balancing
162 heating device
162a heating device
162b heated area
162c unheated area
162d volumes in the heating bag downstream of the annular sensor
163 mixing device
163a conductivity sensor
163b conductivity sensor
165a temperature sensor
165b temperature sensor
166 concentrate supply
167 leakage sensor
168 concentrate supply
169 second flow pump
171 pump, sodium pump
173 pump, bicarbonate pump
300 extracorporeal blood tubing set or blood circuit
301 first line (arterial line section)
302 arterial patient tube clamp
303 blood filter or dialyzer
303a dialysis liquid chamber
303b blood chamber
303c semi-permeable membrane
305 second line (venous line section)
306 venous patient tube clamp
315 air bubble detector; ABD
317 single-needle chamber
318 deaeration device
325 addition site for Heparin
329 (venous) bubble trap
400 effluent bag; filtrate bag
401 dialysis liquid source
bag with dialysis liquid
403 substitute fluid source; bag with substitute fluid
450 temperature measuring device; temperature sensor
500 storage device
A container; A-concentrate; sodium
B container; B-concentrate; bicarbonate
DV conveyed dialysis liquid volume
F1 filter
F2 filter
Pa patient
PS1 arterial pressure sensor (optional)
PS2 arterial pressure sensor (optional)
PS3 pressure sensor (optional)
PS4 pressure sensor for measuring the filtrate pressure (optional)
PS5 pressure sensor for measuring the pressure in the dialysis liquid inlet line
Tmax maximum temperature measured in the measuring interval
t time axis
V valve
V24 valve
V25 valve
VB bypass valves
W scale
Y Y-connector

1-16. (canceled)

17. A blood treatment apparatus comprising:
a fluid line or a connection site configured for a connection to the fluid line,
a pump configured to convey treatment liquid through the fluid line,
a heating device configured to heat the treatment liquid within the fluid line and/or within the heating device,
a temperature measuring device configured to detect, measure, and/or determine the temperature of the treatment liquid using at least one temperature sensor when the treatment liquid is present in the fluid line or in the heating device,
a storage device configured for storing amounts of temperature values or absolute values thereof, and
a control device configured for controlling or regulating the pump configured for conveying treatment fluid,
wherein the control device is programmed to:
prompt the temperature measuring device to measure a plurality of temperature values at a respective plurality of measurement times which are associated with different measurement intervals, and to determine, for each of the measurement intervals, a prominent or maximum temperature value from the temperature values measured during the respective measurement interval;
store the plurality of temperature values, thus determined, in the storage device;
read out the stored, determined temperature values from the storage device;
read out, from the storage device, a preset or predetermined amount or absolute value of the temperature which the treatment liquid at a predetermined site within the fluid line downstream of the heating device can amount to as a maximum value or must at least amount to as a setpoint; and
determine or calculate a maximum conveyance rate of the pump such that the treatment liquid already present downstream of the heating device in the fluid line, the determined temperature values of which have been read out from the storage device, reaches the predetermined site having a temperature lower than or equal to the maximum value, or higher than or equal to the setpoint.

18. The blood treatment apparatus according to claim 17, wherein the pump is a substitute fluid pump.

19. The blood treatment apparatus according to claim 17, wherein the pump for conveying the treatment liquid is a dialysis liquid pump arranged to convey the treatment liquid as dialysis liquid into a dialysis liquid chamber of a blood filter comprising the dialysis liquid chamber and a blood chamber, separated therefrom via a membrane.

20. The blood treatment apparatus according to claim 19, wherein the predetermined site is the dialysis liquid chamber or an inlet into the blood filter.

21. The blood treatment apparatus according to claim 17, wherein the predetermined site is an addition site for treatment liquid, as a substitute fluid, into an extracorporeal blood circuit.

22. The blood treatment apparatus according to claim 17, wherein a length or duration of the measurement intervals is constant and is stored in the storage device.

23. The blood treatment apparatus according to claim 17, wherein amounts or absolute values of quantities of treatment liquid conveyed in each of the measurement intervals are stored.

24. blood treatment apparatus according claim 17, wherein amounts or absolute values of treatment liquid conveyed in each of the measurement intervals are stored together with the respective determined temperature value and a designation for identifying the associated measurement interval corresponding to the respective determined temperature.

25. The blood treatment apparatus according to claim 17, wherein the storage device is or comprises a ring buffer or a ring memory in which the determined temperature values are stored or are to be stored.

26. The blood treatment apparatus according to claim 17, wherein the control device is programmed to limit or otherwise control or regulate a conveyance rate of the pump for conveying treatment liquid such that the temperature of the treatment liquid being conveyed by the pump to the predetermined site does not exceed a predetermined temperature value.

27. The blood treatment apparatus according to claim 17, wherein the control device is programmed to correct or adjust the determined temperature values downwards by a correction factor for periods of time during which the pump is not conveying.

28. The blood treatment apparatus according to claim 17, wherein the control device is programmed to correct or adjust the determined temperature values after storing the determined temperature values.

29. The blood treatment apparatus according to claim 17, further comprising a blood pump.

30. The blood treatment apparatus according to claim 17, wherein the control device is further programmed to determine a cooling of the treatment liquid during passage of the treatment liquid towards the predetermined site.

31. The blood treatment apparatus according to claim 30, wherein determining the cooling of the treatment liquid comprises determining the cooling of the treatment liquid during passage of the treatment liquid between the temperature measuring device and the predetermined site.

32. The blood treatment apparatus according to claim 30, wherein the cooling of the treatment liquid during passage between the temperature measuring device and the predetermined site is determined or at least approximately determined or assumed by one or more look-up tables and/or by calculation.

33. A control device configured for controlling or regulating a pump configured for conveying treatment fluid, wherein the control device is programmed to:
prompt a temperature measuring device to measure a temperature value, respectively, at a plurality of measurement times which are associated with different measurement intervals, and to respectively determine, for each of the measurement intervals, a prominent or maximum temperature value from the temperature values measured during the measurement interval;
store the temperature values, thus determined, in a storage device;
read out the stored, determined temperature values from the storage device;
read out, from the storage device, a preset or predetermined amount or absolute value of temperature which a treatment liquid at a predetermined site within a fluid line downstream of a heating device can amount to as a maximum value or must at least amount to as a setpoint; and
determine or calculate a maximum conveyance rate of the pump such that the treatment liquid already present downstream of the heating device in the fluid line, the determined temperature values of which have been read out from the storage device, reaches the predetermined site having a temperature lower than or equal to the maximum value, or higher than or equal to the setpoint.

34. A digital storage medium with electronically readable control signals, configured to interact with a programmable computer system such that a blood treatment apparatus is programmed or reprogrammed into the blood treatment apparatus according to claim 17.

35. A digital storage medium with electronically readable control signals, configured to interact with a programmable computer system such that a control device is programmed or reprogrammed to the control device according to claim 33.

36. A computer program product configured to interact with a programmable computer system such that a blood treatment apparatus is programmed or reprogrammed into the blood treatment apparatus according to claim 17.

37. A computer program product configured to interact with a programmable computer system such that a control device is programmed or reprogrammed into the control device according to claim 33.

38. A computer program having a program code for prompting the programming or reprogramming of a blood treatment apparatus such that it is programmed or reprogrammed into the blood treatment apparatus according to claim 17.

39. A computer program having a program code for prompting the programming or reprogramming of a control device such that it is programmed or reprogrammed into the control device according to claim 33.
